(19) United States
(12) Patent Application Publication
(10) Pub. No.: US 2023/0305016 A1
(43) Pub. Date: Sep. 28, 2023

(54) TEST KIT AND METHOD OF DETERMINING TRYPTOPHAN IN EXTRACTS OF FAECAL SAMPLES

(71) Applicant: IMMUNDIAGNOSTIK AG, Bensheim (DE)

(72) Inventors: Franz Paul ARMBRUSTER, Bobenheim-Roxheim (DE); Susanne WOLF, Bensheim (DE)

(73) Assignee: IMMUNDIAGNOSTIK AG, Bensheim (DE)

(21) Appl. No.: 18/024,210

(22) PCT Filed: Sep. 2, 2021
(86) PCT No.: PCT/EP2021/074298

§ 371 Date: Mar. 1, 2023

(30) Foreign Application Priority Data
Sep. 2, 2020 (DE) 10 2020 122 984.4
Nov. 3, 2020 (DE) 10 2020 128 969.3

(51) Int. Cl. G01N 33/68

(52) U.S. Cl. CPC G01N 33/6812; G01N 2800/042

(57) ABSTRACT

A method for quantitative determination of bioavailable L-tryptophan in faeces and a method of diagnosis whether a subject is suffering from impaired fructose absorption or from a lack of bioavailable tryptophan. The latter includes the localization of a previously hidden aetiology of decreased blood tryptophan levels and a method of in vitro diagnosis of the aetiology of gastrointestinal (IBD), Crohn's disease, depression symptoms, anxiety, insomnia, sleep disorders, dysphoric disorders. A kit of parts for determining the ratio of free L-tryptophan to blocked glycated tryptophan or fructosyl-tryptophan adduct for immediate treatment of these disorders by providingpatients with appropriate dietary recommendations or an intake of tryptophan that is not blocked or glycated in the acidic environment of the stomach or gastrointestinal tract.

### TECHNICAL FIELD

The invention relates to a method of measuring the concentration of tryptophan involving an immunochemical step, and a test kit for measuring tryptophan in stool and urine (G01N33).

### TECHNICAL BACKGROUND

Tryptophan is an essential amino acid that humans must obtain from the diet. The ingested tryptophan is metabolized to physiologically active neurotransmitters and messengers, including serotonin and kynurenic acid, which regulate pain sensation and recovery, to melatonin, a hormone that regulates the sleep-wake rhythm, niacin also known as nicotinic acid (vitamin B3), NAD and NADP. Methods for determining tryptophan have been described in Methods in Enzymology, XVII, H Tabor and C Tabor Eds. Academic Press, N.Y., 1971 and by Mefford I N & Barchas J D, Chromatogr. 1980, 181:187-193. VanEijk H M et al, in Anal Biochem (1999) 271:8-17 and Fonath A N et al, Amino Acids (2007) 32:213 disclose a determination of tryptophan by HPLC and tandem mass spectrometry, further claimed by U.S. Pat. No. 5,559,038A (Uni Colorado, US) and EP2179282B1 (Quest Diagn. Invest. Inc., US). CN201010300555A (The Second XiangYa Hospital of Central South University) and CN107907603A (Huaren Pharmaceutical Co. Ltd.) disclose a chromatographic separation and fluorescence detection of tryptophan. RU02012869C1 (KAZANSKIJ GOSUDARSTVENNYJ TEK. UNI.) claims photometry of tryptophan using 7-chlor-4,6-dinitrobenzofuroxane as coloring reagent. SU1528394 (NII SELSKOGO KHOZ TS CHERNOZEM) describes a tryptophan determination comprising a hydrolysis step and photometry of tryptophan at 280 nm. WO2014044700A1 (Rupprecht-Karls-Universitat Heidelberg, DE) refers to a determination of a tryptophan degradation product. JPH03160369A (Seiko Instr. Inc.) claims determination of tryptophan wherein a biological sample is adsorbed on a surface and hydrolyzed in situ using mercaptoethanesulfonic acid. CN102323306A (BEIJING XINGYOU FENGKE TECH DEV CO LTD et al) discloses a Belousov-Zhabotinsky oscillation system wherein the change in the oscillation pattern is related to the amount of tryptophan added. SU1394136A1 (NOVOSIBIRSKY G MED I) discloses a determination based on the photo-oxidation constant of tryptophan when hydrogen peroxide is added.

U.S. Pat. No. 4,818,683 (Morel et al.) claims a derivatization of tryptophan to a higher molecular weight antigen followed by a determination based on a binding analysis with an antibody (cf. Ellman G L. Arch Biochem Biophys 1958, 74:443-450; Eberle A & Hübscher W, Helvetica Chimica Acta 1979, 62(7):2460-2483, Riddles P W, Anal Biochem 1979, 94:75-81; Muckemeide A et al, J. Immunol 1987, 138:833; Apple R J et al, J Immunol 1987, 140:290; Domen P J, J Immunol 1987, 139:195; Kobayashi N et al, Advances in Clin Chem 2001, 36:139-170; DE10 2005 060 057A1 (Kellner K H); DE69520383 T2 (Dade Behring Inc.); EP0668504A1 (E.I. DuPont); EP0471345B1 (Boehringer Mannheim GmbH). EP0368271B1 (Ishikawa) describes a biotinylation of tryptophan and EP2612147 (Kellner K H) a derivatization reagent for a speedy binding analysis in an automated process.

Regarding the clinical chemistry of tryptophan, patients with colitis or inflammatory bowel syndrome (IBS) suffer from a more intense sensation of pain when tryptophan levels in the intestinal mucosa are low (Keszthelyi D et al., Decreased levels of kynurenic acid in the intestinal mucosa of IBS patients: Relation to serotonin and psychological state, J Psycho Res 2013, 74(6):501-504). Decreased serum tryptophan levels may also be associated with inflammatory bowel disease (IBD) and Crohn's disease activity (Gupta N K et al., Serum analysis of tryptophan catabolism pathway: correlation with Crohn's disease activity. Inflammatory Bowel Diseases 2012, 18(7):1214-20; Nikolaus S. et al., Increased Tryptophan Metabolism is Associated with Activity of Inflammatory Bowel Diseases. Gastroenterology 2018, 154(6):1855-1856). IBD patients also often have reduced amounts of tryptophan in their stool (Lamas B et al., CARD9 impacts colitis by altering gut microbiota metabolism of tryptophan into aryl hydrocarbon receptor ligands. Nature Medicine 2016, 22(6), 598-605; and Inflammatory Bowel Diseases. 18(7):1214-20).

Fructose malabsorption may also be associated with decreased plasma tryptophan content and symptoms of depression (Ledochowski M et al., Fructose malabsorption is associated with decreased plasma tryptophan, Scand J Gastroenterol 2001, 36 (4):367-71; Ledochowski M et al., Fructose malabsorption is associated with early signs of mental depression. Europ J Med Res 1998, 3(6):295-8). Tryptophan and 5-hydroxytryptophan (5-HTP) are therefore recommended as dietary supplement and mild antidepressant, anxiolytic and sleep aid. The U.S. Institute of Medicine recommended daily allowance (RDA) of tryptophan is 5 mg/kg body weight/day for adults of 19 years and older (Institute of Medicine, Protein and Amino Acids: Dietary Reference Intakes for Energy, Carbohydrates, Fiber, Fat, Fatty Acids, Cholesterol, Protein, and Amino Acids. Washington, DC: The National Academies Press. pp. 589-768). A Cochrane Systematic Review examined whether tryptophan and 5-HTP are effective and safe in alleviating depression in adults and concluded that they are better than placebo but may have serious side effects (cf. Shaw K A et al, Tryptophan and 5-hydroxytryptophan for depression, Cochran Database Syst Rev. 2002, (1): CD003198). However, there is evidence that plasma tryptophan levels are affected only by an oral ingestion of pure tryptophan and not by a tryptophan-enriched diet, which poses a problem for individuals who may have a disturbed tryptophan adsorption or low plasma or serum tryptophan levels. The state of the art represents a problem.

### BRIEF DESCRIPTION OF INVENTION

The problem is alleviated by a method for determining L-tryptophan in faeces of a subject suspected of suffering from dietary fructose intolerance, tryptophan malabsorption, depression symptoms and/or intestinal dysfunction and pains, characterized by the steps of:—
(a) collecting a defined faecal sample of said subject;
(b) extracting the faecal sample in a buffer to prepare an extract of faecal amino acids and soluble faecal compounds;
(c) separating the extract from non-soluble components and preparing aliquots of said extract, one for direct determination of L-tryptophan in said aliquot and one for determination of L-tryptophan in said aliquot after hydrolyzation of glycated tryptophan and sugar-tryptophan adducts;
(d) treating one aliquot of said extract with a strong acid or base to hydrolyze all condensation products of an aldose or ketose with an amino acid or L-tryptophan;
(e) adding to the aliquots a derivatization reagent that reacts with the α-amino groups of amino acids or L-tryptophan to prepare a L-tryptophan derivative;
(f) determining the amounts of L-tryptophan derivative in said aliquots using an antibody specific for L-tryptophan derivative, and;
(g) comparing the amounts of L-tryptophan derivative in the said aliquots to determine the amount and ratio of tryptophan in the faeces as hydrolysable L-tryptophan product subjected to a condensation reaction with an aldose or ketose in the gastrointestinal tract.

In some embodiments, the method comprises the additional step of comparing the amount of hydrolysable L-tryptophan product in the subject's faecal sample to the amount of hydrolysable L-tryptophan product in the faeces of a healthy subject to diagnose a dietary fructose intolerance in the event of an elevated amount of hydrolysable L-tryptophan product. For comparability, the ratio of hydrolysable tryptophan product to tryptophan in faeces may be determined since dietary tryptophan content can vary widely. In some circumstances, it may be helpful to compare the ratio of hydrolysable tryptophan not only with tryptophan but also with other amino acids in the faeces, particularly phenylalanine or leucine. In some cases, it may further be beneficial for diagnosis to simultaneously determine tryptophan and/or hydrolysable tryptophan product in other bodily fluids, particularly plasma, serum, blood, and urine.

In some embodiments, the method comprises localizing the aetiology of a disorder, particularly, when a patient is suffering from digestion disorders, obstipation, constipation, diarrhea, gastrointestinal disorders, colitis, irritable bowel syndrome (IBS), inflammatory bowel disease (IBD), Crohn's disease, dermatitis, depression, insomnia, sleep-wake disorders, migraine, fatigue syndrome, bulimia nervosa, eating disorders, anxiety, dysphoric disorders, burn-out syndrome. This may further comprise a determination of the effective amount of any one of supplementary dietary tryptophan, pharmaceutical composition comprising di- and tripeptides of tryptophan, supplementary dietary niacin (nicotinic acid).

In some embodiments, the method comprises the use of a tryptophan derivatization reagent selected from the group comprising detection reagents with a reactive component, biotin-ε-aminocaproic acid-N-hydroxy succinimide ester, Boc-6-aminocaproic acid N-hydroxy succinimide ester, diiodotyrosine-beta-alanine hydroxysuccinimidyl ester, tryptophan-β-alanine hydroxysuccinimidyl ester. The method then uses an anti-L-tryptophan derivative antibody, and the detector antibody may be conjugated to a detection group, a fluorescent or luminescent dye, an electroluminescent group or an enzyme for detection, a peroxidase.

In some embodiments of the method, the glycated tryptophan or sugar-tryptophan product is hydrolyzed at an elevated temperature between 60 and 100 degrees Celsius in an aqueous solution having a pH between 12 and 14.

Another aspect of the invention relates to kit of parts for determining the amount of hydrolysable glycated tryptophan or sugar-tryptophan adduct, comprising a) a derivatization reagent of tryptophan; b) antibodies which bind a tryptophan derivative; c) a tryptophan derivative as a tracer substance; d) one or more standard solutions of tryptophan and e) sugar-L-tryptophan adduct as hydrolyzation control. In some embodiments, the kit of parts may further comprise one or more microtiter plates wherein the tryptophan-derivative tracer is immobilized on a surface of the wells. The kit of parts may comprise D-fructose-L-tryptophan Amadori product as hydrolyzation control, and a buffer for faeces extraction and for preparing and aqueous solution of aromatic amino acids. The kit of parts may comprise a system for transfer of a defined amount of faeces into a vessel with extraction buffer.

An important aspect of the invention relates to a method of in-vitro diagnosis of dietary fructose intolerance or excessive consumption of fructose, employing a kit of parts as described for determining the amount of hydrolysable tryptophan product in faeces and for diagnosis of a compromised intestinal absorption of fructose or tryptophan or both, preferably in combination with a reduced tryptophan level in the blood, serum, or plasma. The method comprises the collection of a defined faecal sample and its transfer into a vessel containing a stabilization and extraction buffer; a dispersion of the faecal sample and an extraction of the soluble substances into a buffer; a separation of the obtained extract from the solid matrix components and a preparation of first and second aliquots of said extract. A first aliquot will be treated with a strong base to hydrolyse any condensation product or adduct of an aldose or ketose with tryptophan, which is followed by an optional neutralizing step. The measurement of the tryptophan in the first and/or second aliquots are determined by immunological methods, preferably by a competitive immunoassay employing antibodies against a tryptophan derivative and tryptophan derivative as tracer. In brief, by adding to the first and second aliquots a derivatization reagent that reacts with the amino group of tryptophan; by determining the amounts of tryptophan derivative in the first and second aliquots; and by comparing the amounts of tryptophan derivative in the first and second aliquots to determine amount of tryptophan in the faecal sample and the amount of blocked or glycated tryptophan in the faecal sample that has been subject to a condensation reaction with an aldose or ketose in the gastrointestinal tract.

The method of medical diagnosis may lead to a prescription or in an administration of additional pure tryptophan or di- or tripeptides with tryptophan when the faecal sample contains hydrolysable tryptophan products. An administration of pure tryptophan or di- or tripeptides with tryptophan is indicated when the molar ratio of aldose- or ketose-tryptophan adduct to free tryptophan in faeces is greater than 5 percent, in particular greater than 10 percent, and strongly advised when greater than 20% of the free tryptophan in faeces. Alternatively, the patient may be given the dietary advice of avoiding foods and drinks sweetened with large amounts of fructose or fructose-glucose syrup (corn syrup).

In some embodiments, the immunological determination comprises a derivatization of the amino acids and biogenic amines in the extract and antibodies binding the L-tryptophan derivative. The derivatization reagent may be selected from the group comprising:—acylating reagents, ester-activated detection reagents, detection reagents with N-hydroxy succinimide as reactive group, biotin-×-NHS, wherein X is 7-aminocaproic acid or a spacer with up to 24 carbon atoms; Boc-6-aminocaproic acid N-hydroxy succinimide ester, diiodotyrosine-beta-alanine N-hydroxy succinimide ester, tryptophan-β-alanine N-hydroxy succinimide ester and derivatives thereof. In general, the tryptophan may be derivatized to a molecule with one or more haptens. The derivatization reagent may also be coupled via a spacer to a group that is represented by a labeled secondary antibody or a binding protein that can be detected with high selectivity. In general, the derivatization reagent for tryptophan may have the following general formula (I):

R′—(CH₂)ₙ—(CONH)ₘ—(CH₂)ₚ—COOR (I)

wherein R is an activating group, n and p are the same or different and integers from 0 to 12, m is 0 to 4, and R′ is a hapten which can be bound by an antibody or a specific binding protein. Other preferred ester-activated groups are imidazolides, pyridazolides, hydrazides, aminoalkylcarboxylic acids, wherein the alkyl group may have 2 to 24 carbon atoms or activated aryl ester groups such as p-nitrophenyl esters. Haptens containing SH can be reacted with a maleimide derivatization reagent.

In some embodiments, the immunological determination comprises that the targeted tryptophan derivative competes with an immobilized tracer for the binding of the anti-tryptophan antibodies. In some embodiments, a detector antibody is added which binds the anti-tryptophan derivative antibodies, the detector antibody being conjugated to a detection group, a fluorescent or luminescent dye, an electroluminescent group or an enzyme such as peroxidase.

In some embodiments, the immunological detection method may comprise a generation of a response curve of absorbance unit (optical density) versus concentration, using the values obtained from a standard so that the tryptophan derivative present in the sample or aliquot can be determined directly from this curve.

In some embodiments, the condensation products of aldoses and ketoses with tryptophan are hydrolyzed in an aqueous solution having a pH greater than 12, preferably at a temperature between 60 to 100° C. The hydrolyzation of the fructose-tryptophan adduct is time and temperature dependent, but 5 to 10 minutes at 60 to 100 degrees Celsius may give a complete reaction having regard to the excess of hydroxide. A complete hydrolyzation of the sugar-tryptophan adduct can be obtained within 1 to 2 hours.

An aspect of the invention relates to a method of in vitro diagnosis of the aetiology of a deficient tryptophan uptake and/or decreased plasma or serum tryptophan levels. The insufficient tryptophan uptake may be caused by an excessive consumption of foods and beverages sweetened by fructose and fructose-glucose syrups. Such kind of sweeteners are added to many foods and beverages, preferably in the form of processed corn syrup. Fructose-glucose syrup is not only added to sugary soda, candy, ice cream, sweetened yogurt, jam and jelly, juices but is also contained in many salad dressings, frozen junk foods, canned fruit, breads, breakfast cereals, ketchup, dips, and condiments. An insufficient tryptophan absorption may be the aetiology for numerous gastrointestinal disorders, irritable bowel syndrome (IBS), inflammatory bowel disease (IBD), Crohn's disease, pain sensation, depression symptoms, anxiety, insomnia, sleep disorders, dysphoric disorders, fatigue syndrome and/or a compromised immune system. The method of in vitro diagnosis may comprise a determination of the ratio of tryptophan to fructose-tryptophan adduct in faeces in comparison with the ratio found in faeces of healthy subjects.

A further aspect relates to a kit of parts for determining the ratio of tryptophan to fructose-tryptophan adduct in faeces, comprising:
(a) a faeces stabilization and extraction buffer;
(b) a derivatization reagent of extracted amines, selected from Boc-6-aminocaproic acid N-hydroxysuccinimidyl ester, diiodotyrosine-beta-alanine N-hydroxysuccinimidyl ester, tryptophan-β-alanine N-hydroxysuccinimidyl ester to obtain a tryptophan derivative;
(c) antibodies which bind a tryptophan derivative obtained in step (b);
(d) the tryptophan derivative of (b) as a tracer substance;
(e) a buffer for hydrolyzation of adducts of ketoses and aldoses with tryptophan;
(f) one or more standard solutions of tryptophan.

In some embodiments, the kit of parts may comprise a microtiter plate wherein the tryptophan-derivative tracer is immobilized on a surface. The described kit of parts may further comprise a special vial with D-fructose-L-tryptophan as shown by formulae I and II (Amadori/Heyn's product) below:

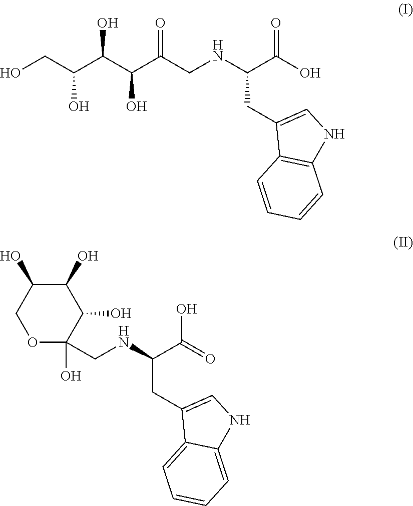 [SMILES: O=C(O)[C@H](Cc1c[nH]c2ccccc12)NCC(=O)[C@@H](O)[C@H](O)[C@H](O)CO ; O=C(O)[C@@H](Cc1c[nH]c2ccccc12)NCC1(O)OC[C@@H](O)[C@@H](O)[C@@H]1O]

In some embodiments, the kit of parts may further comprise a buffer for neutralization of the hydrolyzation reaction prior derivatization.

In some embodiments, the hydrolyzed tryptophan solution is pH adjusted prior derivatization. The hydrolyzed tryptophan solution may be pH-adjusted by addition of HCl conc.

In some embodiments, the extraction and stabilization buffer may contain an excess of 0.001 to 5.0% by weight of ketocarboxylic acid having 1 to 12 carbon atoms which, under ambient conditions, form water-soluble hydrated salts with amino acids and proteins, as well as buffer salts, detergents, notably SDS, solubilizing agents, complexing agents, biocides, ethanol, and adjuvants for stabilization of the faecal sample and for blocking metabolic activity. The later may be traditionally achieved by an addition of triclosan, cresols, phenol, or benzoic acid. The extraction buffer may preferably have a pH between 3 and 6 to facilitate the solution of aromatic amino acids.

The invention will be further described with reference to preferred embodiments, drawings, representative examples and by the claims. The examples and drawings are for illustration only and not be considered limiting in any way. The desired scope of protection has been defined in the claims.

### BRIEF DESCRIPTION OF DRAWINGS

It is shown in: —

FIG. 1 a scheme of the likely gastrointestinal condensation reaction of fructose with tryptophan to form a D-fructosyl-L-tryptophan as well as the Heyn's rearrangement (Amadori rearrangement);

FIG. 2 a bar diagram comparing amounts of tryptophan found in hydrolyzed faecal extracts (shaded bars) and non-hydrolyzed extracts (full bar), prepared from faecal samples of patients suffering from fructose malabsorption and for comparison in extraction buffer (control), which aliquoted extracts have been spiked with 300 μM D-fructose-L-tryptophan Amadori product, 300 μM tryptophan, 300 μM fructose, and 300 μM tryptophan+300 μM fructose;

FIG. 3 a bar diagram comparing the amounts of tryptophan found in hydrolyzed faecal extracts (shaded bars) and non-hydrolyzed extracts (full bars) in faecal samples from patients (no. 3) diagnosed of suffering from fructose malabsorption and of apparently healthy subjects (5)—the white bars show the respective amounts of fructosyl-tryptophan/fructopyranosyl-tryptophan in the sample;

FIG. 4 a bar chart showing the reproducibility of the hydrolysis step (pH 13,2, 100° C., overnight) with respect to the determined amount of tryptophan;

FIGS. 5A, B are representative bar graphs showing the levels of free and treated (free and unblocked) tryptophan over an 8-day period in the stools of healthy subjects who had a 500 mg tryptophan supplement per day and an ad libitum diet which contained more or less fructose.

### DETAILED DESCRIPTION OF INVENTION

### Definitions

The term tryptophan is used hereinbelow for the free amino acid.

The term tryptophan peptide is used for oligopeptides containing tryptophan.

The terms glycated tryptophan and blocked tryptophan shall encompass all tryptophan that has been subject to a condensation reaction with either an aldose or a ketose in the gastrointestinal tract, regardless of whether the product is a Schiff base or underwent an Amadori or Heyn's rearrangement. The term blocked or glycated tryptophan encompasses fructated/fructosylated tryptophan but also other products of a condensation between an aldose and tryptophan, e.g., glucosylated or mannosylated tryptophan.

The term hydrolysable tryptophan product refers to all blocked or glycated tryptophan and sugar tryptophan adducts formed in the stomach and in the lumen of the gastrointestinal tract which set free tryptophan upon a hydrolyzation as described.

The term tryptophan derivative refers to tryptophan which has been subjected in vitro a derivatization using a derivatization reagent to produce a tryptophan antigen.

The term Amadori product of tryptophan describes tryptophan which has been subject to a reaction with an aldose or ketose by a thermal process, e.g., cooking, baking, frying, etc. The Maillard reaction (non-enzymatic browning and glycation) are thought to concern mainly the ε-amino groups of lysine but may also affect peptide-bound tryptophan which can react with reducing carbohydrates. The “sugaramino acids” may be degraded to form 1,2-dicarbonyl compounds which can react further to a multiplicity of so-called “advanced glycation end products (AGEs).

Tryptophan absorbed from the diet is metabolized by the kynurenine pathway and the serotonin pathway. The kynurenine pathway commences with an oxidative degradation of tryptophan to yield nicotinate mononucleotide, a precursor for the biosynthesis of nicotinate nucleotides (NAD and NADP). The serotonin pathway starts either with the tryptophan hydroxylase I in the enterochromaffin cells of the gut or the tryptophan hydroxylase II in the nerve cells of the central nervous system and the brain.

Dietary fructose intolerance (DFI), herein also referred to as fructose malabsorption, is a digestive disorder which affects tryptophan levels in the blood. The dietary fructose intolerance (DFI) must be distinguished from hereditary fructose intolerance (HFI) which is an inborn disease characterized by a deficiency in aldolase B. The dietary fructose intolerance (DFI) is strongly associated with intestinal problems and depression (Ledochowski M et al., Fructose malabsorption is associated with decreased plasma tryptophan, Scand J Gastroenterol 2001, 36 (4):367-71; Ledochowski M et al., Fructose malabsorption is associated with early signs of mental depression. Europ J Med Res 1998, 3(6):295-8). This points to a group of diseases because fructose can normally up-regulate its own absorption in the intestinal tract while the mechanisms for this upregulation are not fully understood. Member 5 (GLUT5) of the glucose transporter proteins, however, is the fructose transporter primarily responsible for the absorption of fructose (Patel C et al, Transport, metabolism, and endosomal trafficking-dependent regulation of intestinal fructose absorption FASEB J. 2015, 29:4046-4058). Fructose has been a part of the human diet since the dawn of time and is contained in many healthy foods such as honey, apples, pears, berries, grapes, and many exotic fruits. Despite its ubiquitous presence, a dietary intolerance to fructose is relatively common in industrialized countries (cf. Lomer M C E., The aetiology, diagnosis, mechanisms and clinical evidence for food intolerance, Aliment Pharmacol Ther 2015, 41:262-275; Berni-Canani R et al., Diagnosing and Treating Intolerance to Carbohydrates in Children, Nutrients 2016, 8(3):157ff). A majority of the patients with irritable bowel syndrome (IBS) even believe that their symptoms are caused by an intolerance to certain carbohydrates [Hammer, H. F. et al., Diarrhea caused by carbohydrate malabsorption, Gastroenterol Clin North Am. 2012, 41:611-627; Zar, S et al., Food hypersensitivity and irritable bowel syndrome, Aliment. Pharmacol. Ther. 2001, 15:439-449). Thus, patients with dietary fructose intolerance seem to fit the profile of patients with irritable bowel syndrome (IBS) and, vice versa, it is known that fructose malabsorption may be caused by intestinal diseases such as celiac disease.

Fructose is absorbed by the enterocytes in the small intestine. Sucrose on the other hand must first be cleaved by sucrase-isomaltase (SI) which is a dual function enzyme, one serving as the isomaltase and the other as sucrose-alpha-glucosidase. The disaccharide sucrose (beet or cane sugar), the commonly called table sugar, is chemically less reactive than fructose or glucose. In crystalline form fructose is a six-membered ring (fructopyranose) and when dissolved it is partly a five-membered ring (fructofuranose) which can readily react with free amines. Fructose is further the most water-soluble of all sugars, and because its sweetening power is 20% higher compared to sucrose, fructose is increasingly used for sweetening of processed foods such as ice cream and soft drinks. Furthermore, fructose can be made on industrial scale from corn starch (maize) which is subjected to immobilized amylase. If the corn syrup is further subjected to a reaction with glucose isomerase a high-fructose corn syrup (HFCS) is obtained which is a popular sweetener for reasons of its low price in addition to palatability and taste enhancement.

The ingested fructose can readily react with amino groups and free amines in the acidic environment of the stomach and in the lumen of the intestines. Corn syrup and other similar high-caloric sweeteners are considered responsible for the increasing prevalence of visceral adiposity, obesity, insulin resistance, diabetes mellitus, metabolic syndrome of the liver, non-alcoholic steatohepatitis (NASH) and nonalcoholic fatty liver disease (NAFLD), and other metabolic disorders (L. Tappy: Fructose-containing caloric sweeteners as a cause of obesity and metabolic disorders. In: The J of Exp Biology 2018, 221, doi:10.1242/jeb.164202). These studies can, however, not clearly distinguish a mechanistic cause because ordinary diets also contain multiple forms of saccharides which are interconvertible in the body and share many steps of the carbohydrate metabolism pathways. Not yet studied has been whether a consumption fructose can affect tryptophan absorption and interferes with the generation of physiologically relevant substances serotonin, kynurenic acid and melatonin. It has further not been studied whether fructose favors diseases associated with a reduced tryptophan absorption and low tryptophan levels in blood, namely gastrointestinal problems, intestinal diseases and disorders, depression, dysphoric disorders, insomnia, sleep disorders, burn-out, fatigue syndrome or a suppressed or compromised immune system.

The relationship between fructose malabsorption and tryptophan malabsorption is puzzling because the enterocytes of the intestine are endowed with a suite of broadly specific amino acid transporters on their apical membranes. There are transporters for neutral amino acids, cationic amino acids, anionic amino acids, imino acids, and p-amino acids. A different set of transporters is found in the basolateral membrane, allowing amino acids to be released into the blood stream after nutrient intake. Expression levels of these transporters are high in the small intestine, where the bulk of nutrient absorption occurs, and they normally ensure an efficient removal of all groups of amino acids from the lumen of the intestine. While not wishing to be bound by any theory, the inventors believe that in the acidic environment of the stomach and the lumen of the intestines aldoses and ketoses (monosaccharides) can form an adduct with L-tryptophan by a nucleophilic reaction. Common natural aldoses are glyceraldehyde, erythrose, threose, ribose, arabinose, xylose, lyxose, allose, altrose, glucose, mannose, gulose, idose, galactose, talose. The aldoses can tautomerize to ketoses, which is a reversible reaction, so that aldoses and ketoses are to some extent in equilibrium with each other. Common ketoses in food are dihydroxyacetone, erythrulose, ribulose, xylulose, psicose, fructose, sorbose, tagatose. All ketonic monosaccharides are reducing because they can tautomerize into aldoses, and the resulting aldehyde group can be oxidized. The most important monosaccharides in the human diet are glucose (an aldose) and fructose (a ketose). It is noted that ketoses are chemically more reactive than the aldoses so that the condensation reaction occurs with fructose at a faster rate than with glucose (Kato H et al., Mechanisms of browning degradation of D-fructose in special comparison with (o-glucose-glycine reaction. Agric Biol Chem 1969, 33:939-48; Mauron J. The Maillard reaction in food: a critical review from the nutritional standpoint. Prog Food Sci 1981, 5:5-35).

The condensation of fructose with an amino group results in a Schiff base which can cyclize to a glycosylamin. The glycosylamin may then undergo a rearrangement, which has been named Heyn's rearrangement in the case of fructose. There are reports that a condensation between protein amino groups with fructose also occur in vivo (McPherson J D et al., Role of fructose in glycation and cross-linking of proteins. Biochemistry 1988, 27:1901-7; Suárez G et al., Administration of an aldose reductase inhibitor induces a decrease of collagen fluorescence in diabetic rats. J Clin Invest 1988, 82:624-7; Walton D J et al., Fructose mediated cross-linking of proteins in: Baynes J W, Monnier V M, eds. The Maillard reaction in aging, diabetes, and nutrition. New York: Alan R Liss, 1989:163-70; Suarez G et al.: Nonenzymatic glycation of bovine serum albumin by fructose (fructation). Comparison with the Maillard reaction initiated by glucose. In: J Biol Chem 1989, 264(7):3674-3679).

The reaction between tryptophan and fructose in the gastrointestinal tract and stomach however has not yet received attention in the scientific literature as there was no way to test this. In any case, this reaction results in a glycated tryptophan (fructosyl tryptophan, fructopyranosyl tryptophan or fructofuranosyl tryptophan) which may be subject to a further rearrangement. The Heyn's and Amadori products of fructose and glucose must therefore be hydrolyzed before the amino acid can be taken by the amino acid transporters of the enterocytes of the small intestine. This is no physiological reaction in the stomach. It is further difficult to determine the formed glycated tryptophan or fructosyl-tryptophan in the small intestine. The inventors have conceived therefore a method to determine the content and the ratio between tryptophan and glycated tryptophan in faeces. It seems obvious to determine in parallel or separately the content and ratio of tryptophan and glycated tryptophan in blood (plasma, serum) and in the urine of patients to localize the aetiology of disorders and a tryptophan/fructose malabsorption.

The intestinal absorption of fructose can be measured using the hydrogen breath test. When fructose is not absorbed, it is anaerobically fermented in the large intestine by the colonic flora. The formed hydrogen is transported to the lungs, where it is exchanged across the lungs and is measurable by the hydrogen breath test. The colonic flora also produces carbon dioxide, short-chain fatty acids, organic acids, and trace gases in the presence of unabsorbed fructose and generate gastrointestinal symptoms such as bloating, diarrhea, flatulence, and gastrointestinal pain. There is no such test available for glycated amino acids, notably fructated tryptophan.

The well-studied Maillard reaction (non-enzymatic browning and glycation) concerns mainly the ε-amino groups of lysine which can also react with reducing carbohydrates. The resulting “sugaramino acids” are degraded during prolonged heating to 1,2-dicarbonyl compounds which are attacked nucleophilic by amino acid side chains so that peptide-bound glycation compounds are formed, so called “advanced glycation end products (AGEs). Besides lysine derivatives also arginine derivatives and crosslink-amino acids (e.g., pentosidine) have been described. Much less is known about the bioavailability and metabolic transit of dietary glycation compounds except that the Amadori products of lysine are NOT absorbed in the intestine and NO source of lysine (“blocked lysine”). The glycated lysine has a non-physiological form which is “biologically unavailable” (see Finot P A et al., Availability of the true Schiffs bases of lysine. Chemical evaluation of the Schiffs base between lysine and lactose in milk. Adv Exp Med Biol. 1977; 866:343-65). This is because the amino acid transporters in the small intestine cannot transport glycated amino acids. The non-transportable glycated lysine is however degraded by the intestinal microbiota. The metabolism of 14C-labeled amino acid Amadori compounds (leucine, phenylalanine, and lysine) has been studied in rats and is summarized in Table 1 below (Finot P A, The Absorption and Metabolism of Modified Amino Acids in Processed Foods, J AOAC INT 2005, 88(3):894-903 and references therein). The urinary excretion was found to be of the order of 60% for Amadori derivatives of leucine, 70% for phenylalanine, 60% for tryptophan and 20.3% for ε-lysine derivatives.

TABLE 1
Metabolism of the Amadori compounds of free amino acids in rats a)

| Amadori | Urinary | Fecal | Retention | Retention | Oxidation |
|---|---|---|---|---|---|
| compounds of | excretion | excretion | in liver | in kidneys | into ¹⁴CO₂ |
| ¹⁴C-Leucine | 60 | n.d. | 1.5 | | x^{b} |
| ¹⁴C-Tryptophan | 60 | n.d. | 0.7 | | xx^{b} |
| ¹⁴C-Phenylalanine | 70 | n.d. | 0.3 | | 10.4 |
| ¹⁴C-ε-Lysine | | | | | |
| ingested (27 h) | 19.5 | 7.1 | 3.2 | 0.6 | 35.2 |
| I.V. injection (8 h) | 53.2 to 62.3 | 0.1 to 0.4 | 1.1 to 1.2 | 5.2 to 6.6 | 1.8 to 2.3 |
| ε-Lysine | 60.0 to 72.2 | 2.5 to 26.4 | | | |

a) Values are expressed in % of the ingested/injected material.
^{b}Detected but not quantified.

A Schiff base from lysine and a ketose (e.g., fructosyl-lysine) is 100% bioavailable in rats because hydrolyzed in the acidic pH of the stomach. However, in case of a rearrangement a fructose-lysine or fructose-tryptophan adduct will likely be acid stable and remain a non-transportable glycated adduct. The concentration of glycated tryptophan in faeces can therefore be used for diagnosis of a dietary fructose intolerance as well as for a localization of the aetiology of disorders caused by a lack of bioavailable tryptophan. An increased amount of non-transportable glycated tryptophan in faeces can therefor stand for either a fructose malabsorption or an absence or shortage of expressed fructose transporter or an excessive consumption of fructose-enriched food or any combination thereof.

As mentioned, the transporter protein GLUT5 is required for intestinal fructose absorption, and its expression is induced in the intestine and in the skeletal muscle of type 2 diabetes patients. On the other hand, its expression is under the transcriptional control by the liver X receptor a (LXRα, NR1H3) because mice treated with LXR agonist T0901317 show an increase in GLUT5 mRNA and increased protein levels in duodenum and adipose tissue. Thus, it can be assumed that fructose absorption in the intestine is highly regulated in humans too.

With respect to tryptophan, the epithelial cells of the small intestine have amino acid transporters on their apical membrane which actively absorb groups of amino acids from the lumen of the intestine. These transporters always bind a range of amino acids rather than individual amino acids. There is another set of these amino acid transporters in the basolateral membrane of these epithelial cells for a release of transferred amino acids into the blood stream. The amino acid transporters could be identified via some inherited disorders such as cystinuria, lysinuric protein intolerance, Hartnup disorder, iminoglycinuria, and dicarboxylic aminoaciduria. The transporter systems have been named in accordance with their amino acid preference: system L (leucine) for large hydrophobic neutral amino acids; system A (alanine) for small and polar neutral amino acids; system ASC for alanine, serine, and cysteine; system N for asparagine, histidine, glutamine, and system T (tryptophan) for aromatic amino acids (tryptophan, phenylalanine). System L and system T can both take tryptophan since tryptophan depletion is thought to be underlying most if not all clinical symptoms observed in Hartnup disorder, which is a renal aminoaciduria resulting from an inherited disorder of neutral amino acid transport (system L). The pellagra-like skin rash, which is typical for the Hartnup disorder, seems to result from a deficiency of nicotinamide/nicotinic acid (niacin), which is mainly synthesized from tryptophan. The rash can be treated by niacin supplementation and an administration of tryptophan-containing dipeptides which uptake and adsorption are mediated by another transporter in the human intestine, the oligopeptide transporter (PEPT1). In vivo and in vitro studies of the PEPT1 oligopeptide transporter have shown that it transports dipeptides and tripeptides only but not free amino acids or peptides with more than three amino acid residues. Tryptophan-containing dipeptides can therefore normalize plasma levels of tryptophan and reduce pain sensation and IBS colitis to normal levels. The relevance of bioavailable tryptophan in the diet is therefore obvious and represents a rational for an administration of pure tryptophan and/or tryptophan-containing di- and tripeptides in the treatment of gastrointestinal disorders provoked or worsened by fructose malabsorption, fructose oversupply or insufficient tryptophan supply.

The quantitative determination of L-tryptophan in faeces commences with the step of (a) collecting and transferring a defined faecal sample into a vessel containing an extraction buffer. The extraction buffer may contain chaotropic substances, buffer salts, a detergent such as Tween® or SDS for solubilization and dispersion. The pKa of tryptophan is 2,38 for the carboxyl group and 9.39 for the amino group. Tryptophan is therefore best dissolved in a buffer having a pH between 3.0 and 6.0; The next step is (b) an extraction of the soluble substances from the faecal matrix, followed by (c) a separation of the extract from the solid components. First and second aliquots of said extract are prepared and (d) a first aliquot is treated with a strong base to hydrolyze any condensation product of an aldose or ketose with tryptophan, optionally followed by a neutralizing step. The hydrolyzation is preferably done in a basic solution by an addition of concentrated NaOH or KOH, e.g., 10 M NaOH, to achieve a pH between 12 and 14 at 60 to 100 degrees Celsius. After readjusting the pH (e) a derivatization reagent is added that reacts with the α-amino group of tryptophan. This is followed by (f) a determination of the amounts of tryptophan derivative in the first and second aliquots, and by (g) comparing the amounts of tryptophan derivative in the first and second aliquot the amount of bioavailable tryptophan in the faecal sample is determined. The amount of non-transportable blocked tryptophan in the faecal sample represents the proportion of tryptophan that has been subject to a condensation reaction with an aldose or ketose in the gastrointestinal tract.

The amounts or concentrations of tryptophan derivative in the first and/or second aliquots are preferably determined by a competitive assay employing antibodies against the tryptophan derivative and tryptophan derivative as tracer. Such an assay requires a derivatization of the amino acids and amines in the faecal extract. The derivatization reagent may be selected from the group comprising:—acylating reagents, detection reagents comprising as reactive component a N-hydroxy succinimide group, biotin-X-NHS (biotin-ε-aminocaproic acid-N-hydroxy succinimide ester), biotin-X-NHS, wherein X is 7-aminocaproic acid or a spacer with up to 24 carbon atoms; Boc-6-aminocaproic acid N-hydroxy succinimide ester, diiodotyrosine-beta-alanine N-hydroxy succinimide ester, tryptophan-β-alanine N-hydroxy succinimide ester and derivatives thereof. In general, the L-tryptophan may be derivatized to a molecule with one or more haptens. The derivatization reagent may be coupled via a spacer to a group that is represented by a labeled secondary antibody or a binding protein that can be detected with high selectivity. In general, the derivatization reagent for tryptophan may have the following general formula (I):

R′—(CH₂)ₙ—(CONH)ₘ—(CH₂)ₚ—COOR (I)

wherein R is an activating group, n and p are the same or different and integers from 0 to 12, m is 0 to 4, and R′ is a hapten which can be bound an antibody, or a specific binding protein. The preferred ester-activated groups are N-hydroxyester groups such as the hydroxysuccinimidyl group, imidazolides, pyridazolides, hydrazides, aminoalkylcarboxylic acids, wherein the alkyl group may have 2 to 24 carbon atoms or activated aryl ester groups such as p-nitrophenyl esters. Haptens containing SH can be reacted with a maleimide derivatization reagent.

The immunological determination may comprise the use of an immobilized tracer, the tracer being bound at a microtiter plate or beads. The L-tryptophan derivative will then compete with the immobilized tracer for the binding of the anti-L-tryptophan antibodies. The detector antibody may be conjugated to a detection group, a fluorescent or luminescent dye, an electroluminescent group or an enzyme such as peroxidase. The evaluation comprises as well known in the art a generation of a response curve of absorbance unit (optical density) versus concentration, using the values obtained from a standard so that the L-tryptophan in the sample or aliquot can be determined directly from this curve.

The medical treatment may comprise an administration of pure tryptophan when the faecal sample contains more than 25 nM per gram stool hydrolysable tryptophan, say tryptophan that has been subject to a condensation with an aldose or ketose (aldose- or ketose-tryptophan adduct). Alternatively, an administration of pure tryptophan is indicated when the ratio of aldose- or ketose-tryptophan adduct to tryptophan is greater than 10 percent, particularly greater than 20% of the free tryptophan. Alternatively, a di- or tripeptide of tryptophan may be administered, preferably in combination with niacin or nicotinic acid, when the concentration of tryptophan in blood (plasma or serum) is below normal levels, say below levels observed in healthy subjects. Alternatively, the medical treatment may consist in a dietary advice of avoiding food and drinks that contain high amounts of fructose. The medical treatment will depend of course on the patient's symptoms as well as on his or her tryptophan levels in plasma or serum.

The medical treatment can take account of the aetiology of an abnormal tryptophan uptake and/or decreased tryptophan levels in the blood. This can be achieved as described above by comparing the amounts of tryptophan in the first and second aliquots of the faecal extract to determine the amount or proportion of tryptophan faeces which had reacted with an aldose or ketose in the gastrointestinal tract and had therefore become biologically unavailable or blocked. When the proportion of blocked tryptophan in faeces is high in comparison with faeces from healthy subjects, the aetiology can be localized to a dietary fructose intolerance or, in the alternative, an excessive ingestion of aldoses and ketoses. More precisely, the aetiology of an abnormal tryptophan uptake may be of dietary origin and a consumption of processed food containing high amounts of fructose, notably in the form of fructose-glucose syrups. High fructose glucose syrups (corn syrups) are commonly added to many foods and beverages such as sweet sugary soda, candy, ice cream, sweetened yogurt, juices, jam, and jelly but also in salad dressing, frozen junk foods, canned fruit, breads, breakfast cereals, ketchup, dips, and condiments. This may be hidden aetiology of a plethora of gastrointestinal disorders, including irritable bowel syndrome (IBS), inflammatory bowel disease (IBD), Crohn's disease as well as for plasma tryptophan dependent disorders, including depression symptoms, anxiety, insomnia, sleep disorders, dysphoric disorders, fatigue syndrome and/or a compromised immune system. The in vitro diagnosis may be based for reasons of comparison and standardization on the ratio of “free” tryptophan to blocked tryptophan (glycated tryptophan, fructosyl-tryptophan adduct) in faeces and a comparison of said ratio with the ratio found in faeces of healthy subjects.

The kit of parts for determining combined tryptophan and blocked tryptophan (glycated tryptophan, fructose-tryptophan adduct, sugar-tryptophan adduct, AGE of tryptophan) in faeces will be supplied preferably for a competitive assay such as an ELISA with following components: microtiter plate, pre-coated and ready-to-use; wash buffer concentrate, tryptophan standards, controls for tryptophan and “blocked tryptophan”; faeces extraction buffer and solvent for aromatic amino acids, anti-L-tryptophan antibody (derivative), detection antibody, conjugated with a marker or enzyme; derivatization reagent and solvent therefore (e.g. DMSO), assay buffer, stop solution, and detection solution (e.g. tetramethylbenzidine). The derivatization reagent may be selected from Boc-6-aminocaproic acid N-hydroxysuccinimidyl ester, iodotyrosine-beta-alanine N-hydroxysuccinimidyl ester, tryptophan-β-alanine N-hydroxysuccinimidyl ester. The control for blocked tryptophan is preferably a D-fructose-L-tryptophan Amadori product as shown in formulae I and II below:

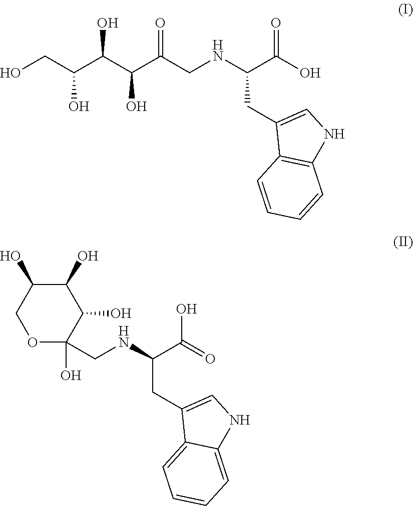 [SMILES: O=C(O)[C@H](Cc1c[nH]c2ccccc12)NCC(=O)[C@@H](O)[C@H](O)[C@H](O)CO ; O=C(O)[C@@H](Cc1c[nH]c2ccccc12)NCC1(O)OC[C@@H](O)[C@@H](O)[C@@H]1O]

The kit of parts may further comprise a buffer for neutralization of the hydrolyzation reaction prior derivatization. The hydrolyzed tryptophan solution may be pH-adjusted by an addition of concentrated HCl conc.

### Example 1

### Test Principle

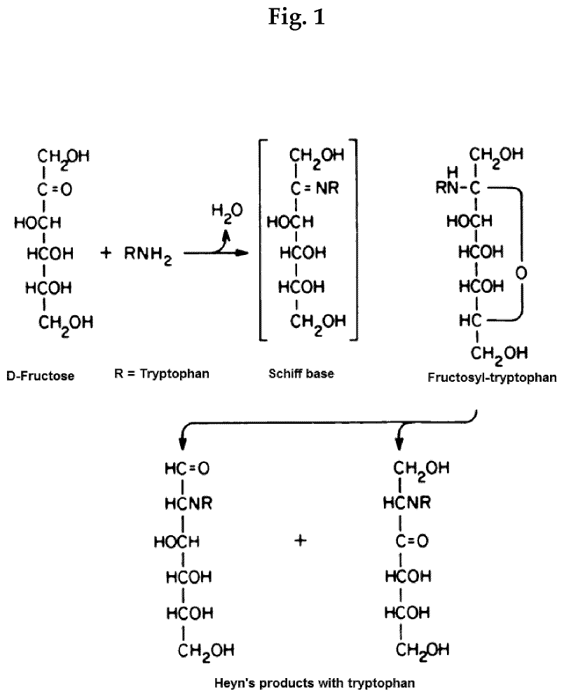

The amino group of tryptophan can react with fructose and other monosaccharides to form a fructose-tryptophan adduct as shown in FIG. 1. The fructose-tryptophan adduct can undergo a so-called Amadori or Heyn's rearrangement to form a stable glycated amino acid. Since intestinal enterocytes do not possess a transporter for fructosylated or glycated tryptophan at their apical membrane and since glycation compounds and advanced glycated end products (AGEs) are “generally” not absorbed by the body, glycated tryptophan in faeces can serve as a biomarker for dietary fructose malabsorption and for localizing the aetiology of gastrointestinal problems and other diseases associated with decreased tryptophan levels in plasma, serum, or blood. For this purpose, random fecal samples from patients suffering from fructose malabsorption and from healthy volunteers were analyzed for their content of tryptophan and glycated tryptophan/fructose-tryptophan adduct.

### Quantitative Determination of Tryptophan

Quantitative determination of L-tryptophan was performed using a commercial competitive enzyme-linked immunoassay (Immundiagnostik AG, Art. No. K7729, Bensheim). Briefly, 15 mg stool was extracted in 750 μL IDK® Amino Acid Extraction Buffer, pH 3,5 (Immundiagnostik AG, Art. No. K7999.100) using the Stool Sample Application System—SAS (Art. No. K6998SAS) to obtain a final stool sample dilution of 1:50.

The tryptophan preparation was completed by adding 50 μL of derivatization reagent (100 mg biotin-ε-aminocaproic acid-N-hydroxy succinimide ester dissolved in 6 mL DMSO) to the stool extract diluted in 250 μL of assay buffer (dilution: 1:13). Derivatization was done at room temperature on a shaker for 45 minutes.

50 μl derivatized standards/controls/samples/hydrolyzed samples were determined as duplicates. The samples and a polyclonal L-tryptophan antiserum were incubated in the wells of a microtiter plate coated with L-tryptophan derivative (tracer). During the incubation period, the L-tryptophan in the sample competes with the tracer immobilized on the wall of the microtiter wells for the binding of the polyclonal antibodies. During the second incubation step a peroxidase-conjugated antibody was added to each microtiter well to detect the L-tryptophan antibodies. After washing off unbound components tetramethylbenzidine (TMB) was added as a peroxidase substrate. The enzymatic reaction was terminated by an acidic stop solution (H₂SO₄ conc). The color changed from blue to yellow and the absorbance was measured in a photometer at 450 nm. The intensity of the yellow color is inverse proportional to the tryptophan concentration in the sample; this means, high L-tryptophan concentration in the sample reduces the concentration of tracer-bound antibodies and lowers the photometric signal. A dose response curve of the absorbance unit (optical density, OD at 450 nm) vs. concentration was generated, using the values obtained from the standards. L-Tryptophan, present in the patient samples, could be determined directly from this curve. The results were multiplied by the dilution factor (15 mg stool in 750 μL extraction buffer=1:50×1:13=1:650) to arrive at the concentration of tryptophan in stool (1 μmol/L˜1 nmol/g stool 0,2 μg tryptophan/g stool; molar mass of tryptophan=204 Da).

### Hydrolyzation of Glycated Amino Acids

70 μl stool extract was combined with 10 μl aqua dest. or spiking solution. For hydrolyzation of the glycated tryptophan/fructose-tryptophan adduct, the extract was adjusted to pH 12 by 10N NaOH and incubated at 100° C. for 20 minutes to hydrolyze any glycated tryptophan and fructose-tryptophan adduct. The hydrolyzed aliquot was again neutralized by 10 N HCl conc., now containing a combined tryptophan concentration of free and released tryptophan from glycated adduct (dilution=1:1,3)

25 μl hydrolyzed stool extract was diluted in 175 μl assay buffer and derivatized with 50 μl derivatization reagent (100 mg biotin-ε-aminocaproic acid-N-hydroxy succinimide ester dissolved in 6 mL DMSO) at room temperature for 45 minutes on a shaker (dilution 1:10). The tryptophan determination was then again performed as described above. The results were multiplied by the dilution factors (1:50×1:1,3×1:10=650).

### Spiking Experiments

5 mg fructose-tryptophan Amadori product (MW=366.37 Da) was dissolved in 2.27 ml aqua dest. (6 mM/L). The concentration of peptide groups in this solution was determined to be 4.36 mM/L using a BCA test (Smith P K et al, Measurement of protein using bicinchoninic acid, in: Analytical Biochemistry 1988, 150(1):76-85, doi:10.1016/0003-2697(85)90442-7). This concentration was taken in the spiking experiments. The IDK® Amino Extract (dilution 1:50) was spiked with fructose-tryptophan Amadori product (not the raw stool) to achieve a target concentration the spiked fecal extract of 6 μM Amadori adduct/L. This corresponds to a spiking of 300 μM fructose-tryptophan Amadori product per gram stool as the stool was diluted 1:50 in the extract.

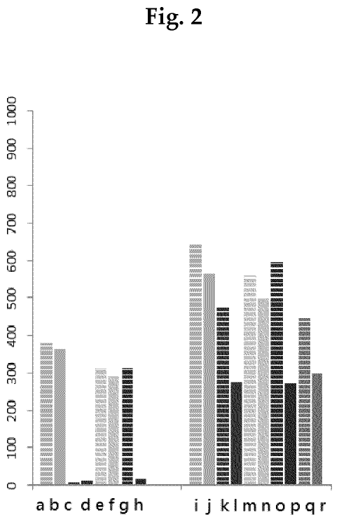

The spiking and hydrolyzation was carried out as follows: 70 μl stool extract were spiked with 10 μl 60 μM/L fructose-tryptophan Amadori product (as determined by BCA) and 10 μl 10 M NaOH. This solution was incubated at 100° C. for 20 min. to achieve hydrolyzation of all adducts and centrifuged at 550 rpm for removal of any precipitate. Finally, 10 μl 10 M HCl was added to neutralize (dilution 1:10) the solution prior measurement of tryptophan. In case of a spiking with fructose or tryptophan, the spiking solution contained 60 μM/L fructose and/or tryptophan each. The results are shown in FIG. 2. They show that added fructose-tryptophan Amadori product is hydrolyzed and that the quantitative measurements are not disturbed or inhibited by added fructose or tryptophan.

### Example 2

Determination of Tryptophan in Faeces from Patients Suffering from Fructose Malabsorption

With reference to FIG. 2, extracts of faecal samples (15 mg) from patients suffering from fructose malabsorption were prepared (samples (i) through (r)) as described in Example 1. The tryptophan contents in the extracts were determined as described above prior (sample r) and after (sample q) hydrolyzation, after spiking of the extract with 300 μM D-fructose-L-tryptophan of formula II (samples p and o), 300 μM L-tryptophan (n+m), 300 μM fructose (l+k), and 300 μM fructose and 300 μM tryptophan (samples j+i). The spiking is calculated on the amount of faecal sample in grams. The shaded/belted bars refer to the tryptophan found in the faecal sample after hydrolyzation. The fecal samples a through h are controls spiked with 300 μM fructose-L-tryptophan adduct (h+g), 300 μM tryptophan (f+e), 300 μM fructose (d+c) and 300 μM tryptophan+300 μM fructose (a+b). The results shown in FIG. 2 confirm the feasibility of the method and that fructosyl-tryptophan is present in faeces of patients suffering from fructose malabsorption.

### Example 3

Comparison of Faeces from Healthy Subjects and Subjects Suffering from DFI

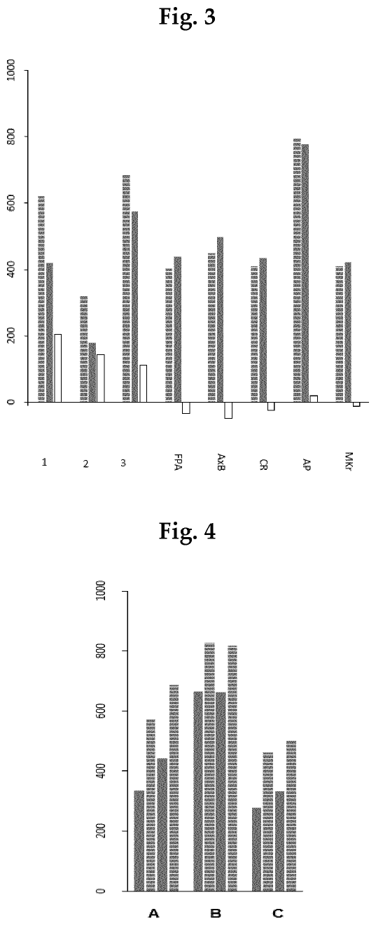

FIG. 3 shows the results of tryptophan analyses of faecal samples from healthy subjects (initials) and patients suffering from fructose malabsorption (samples 1-3). The belted bars refer to the tryptophan determined after hydrolyzation of glycated tryptophan, the full bars to the tryptophan prior hydrolyzation of glycated tryptophan, and the open bars the difference between the two measurements.

The results in the diagram of FIG. 3 show that faeces of patients suffering from fructose malabsorption contained glycated tryptophan and that these patients may therefore suffer from decreased tryptophan absorption. As discussed above, a decreased tryptophan absorption can lead to insomnia or poor sleep-wake rhythm (melatonin deficiency), depression (serotonin deficiency), dysphoria, burn-out, fatigue syndrome, suppressed immune system (niacin, nicotinic acid, vitamin B3 deficiency), and gastrointestinal problems (absence or lack of tryptophan at the brush border).

The amounts of glycated tryptophan adduct in faeces of apparently healthy subject were negligible. This finding suggests that the glycated tryptophan in the faeces of subjects suffering from fructose malabsorption was ingested with the food but was formed in the acidic environment of the stomach and the lumen of the gastrointestinal tract.

FIG. 4 shows duplicate determinations of three different faeces (A-C) from patients with fructose malabsorption or gastrointestinal problems. The results confirm that the hydrolysis method as well as the subsequent tryptophan determination in extracted stool are very highly reproducible.

It remains to be investigated whether the consumption of foods and beverages with high fructose content, e.g., sweetened with fructose or fructose-glucose syrup, has a substantive effect on bioavailable tryptophan in the gastrointestinal tract. As discussed earlier, the concentration of fructose transporters in the gastrointestinal tract, as well as fructose absorption, is physiologically controlled by feedback mechanisms and depends on the diet whereas tryptophan is absorbed by largely specific amino acid transporters. Thus, monosaccharides and, in particular, high concentrations of fructose in the acidic environment of the stomach can cause tryptophan to form reversibly a Schiff base with fructose, subsequently forming a glycated tryptophan which neither be transported nor absorbed. Consequently, glycated or blocked tryptophan in the faeces of patients is a biomarker for a variety of diseases that can be treated either by a changed diet or by supplementation such as pure tryptophan or di- and tripeptides of tryptophan absorbed by a different transporter (PEPT1).

### Example 4

Determination of Free and Blocked Tryptophan in Faeces from Healthy Subjects Receiving a Pure Tryptophan Supplement

Informed healthy volunteers (10) were asked to take a capsule containing 500 mg of tryptophan supplement every evening and to carefully sample their stools over a period of one week (8 days) so that the levels of free and blocked tryptophan could be determined in them. They were also asked to keep a diary of their food intake and note any body changes.

Extraction of the stool matrix: Using a stool sample preparation system filled with amino acid extraction buffer (IDK™ Amino Extract—Immundiagnostik AG, Bensheim, DE-Art. No. K 7999), 15 mg of crude stool samples were collected in each case. The stool was suspended in 750 μl extraction buffer (IDK™ Amino Extract) and its matrix extracted for 10 minutes (dilution 1:50). The extracts were transferred to 1.5 mL Eppendorf tubes, solids pelleted by centrifugation, and 200 μL of the supernatant was transferred to a sealable Eppendorf reaction tube for alkaline treatment. The alkaline treatment was performed in 0.6 M NaOH in extraction buffer: 200 μL of supernatant (stool extract) was mixed with 60 μL of 0.6 M NaOH (pH >12) and heated at 98° C. for 20 minutes. The treated extract was neutralized with 60 μL of 0.6 M HCl (final dilution 1:80). 25 μL of treated and untreated extract were each derivatized for immunological determination of free or treated (free and unblocked) tryptophan content. Quantitative determination of L-tryptophan was performed as described in Example 1 using the IDK® Tryptophan highly sensitive ELISA (Immundiagnostik AG, Bensheim, DE-Art. No. KR3730). Tryptophan concentrations were determined in the untreated and treated stool extracts and the determined concentrations normalized according to the dilution factors. The following were determined for each stool extract collected over the test period: the concentration of free tryptophan, of blocked tryptophan, the difference between free and blocked tryptophan, and the percentage of tryptophan blockage (fructosylated tryptophan and hydrolysable Amadori products with tryptophan) relative to free tryptophan in stool.

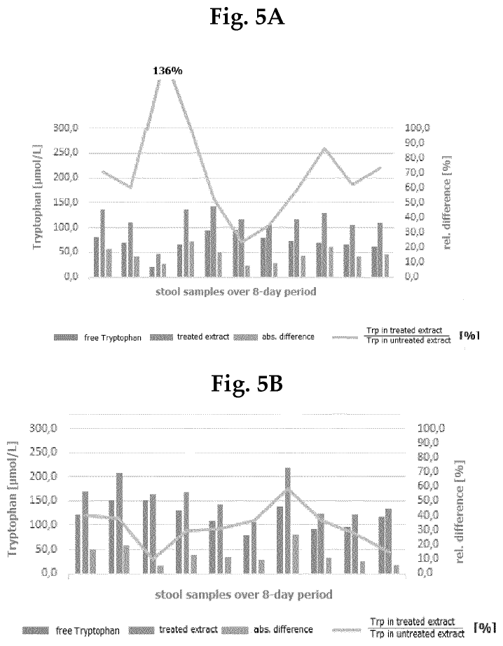

The results have been summarized in FIGS. 5A and B. The determined concentrations of free and blocked tryptophan in the collected stools (n=80 stool extracts) were highly variable which also reflects the ad libitum food and food intake by the volunteers. Their diets were not restricted to a specifically fructose-rich diet. The tryptophan concentration varied in the untreated stool extracts from ˜20 to ˜240 μmol tryptophan/L (average value ˜120 μmol/L) and in the treated extracts from ˜50 to 300 μmol tryptophan/L. The relative amounts of free to blocked tryptophan in stool ranged from 6 to 132 percent (based on the amount of free tryptophan), which means that in some stool samples the concentration of blocked tryptophan was more than twice that of free tryptophan. This also means that a considerable proportion of the tryptophan in the diet could not be absorbed and metabolized into physiologically the active neurotransmitters such as serotonin or melatonin.

The striking day-to-day as well as the diurnal variation of free to blocked tryptophan in the stool confirms the impact of a fructose-rich food on the individual's tryptophan balance, cf. FIGS. 5A and B. On the other hand, the diurnal variation of depressive symptoms appears to be part of the core of depression. Morning lows, afternoon slump, evening worsening—all can occur during a single depressive episode. Mood variability, or the propensity to produce mood swings, appears to be the characteristic that most predicts capacity to respond to treatment. While supplementation of tryptophan and 5-HTP are effective and safe in alleviating depression in adults according to the Cochran review, the present findings suggest that the fructose intake and, of course, a diagnosed fructose malabsorption can offset a tryptophan-rich diet or supplementation with pure tryptophan or 5-HTP. The relative variation of free tryptophan in feces and the results shown in FIGS. 5A and B indicate that the large day-to-day and diurnal variations of free to blocked tryptophan in stool are related to the particular foods consumed and especially to their fructose contents. The fructose content in foods consumed has an impact on mood and sleep as well as on physiological variables such as core body temperature. Small shifts in the ratio of free to blocked tryptophan in the gastrointestinal tract are therefore likely associable to mood state, sleep and sleep phase, depressive symptoms, and numerous gastrointestinal disorders. The present invention therefore provides a suitable means for treating these disorders since many patients do not, and often cannot, know the fructose content of their diet, and thus cannot overlook what is causing or aggravating their symptoms.

### Synopsis

The present application discloses a method of in-vitro diagnosis of dietary fructose intolerance and/or the hidden etiology of numerous gastrointestinal disorders, irritable bowel syndrome (IBS), inflammatory bowel disease (IBD), Crohn's disease, depression symptoms, anxiety, insomnia, sleep disorders, dysphoric disorders, lack of appetite, etc. These disorders are often caused by a lack of bioavailable tryptophan at the intestinal brush and/or insufficient tryptophan absorption, even in case of a tryptophan-enriched diet. The intrinsic biomarker is the amount of blocked tryptophan in the gut and faeces. Tryptophan can be blocked in the acid environment of the stomach by a nucleophilic reaction with dietary aldoses and ketoses. Glycated tryptophan may, in addition, undergo some rearrangements. The amount of blocked tryptophan can be determined by comparing the amounts of tryptophan in faecal extracts without and after hydrolysis of the blocked tryptophan (glycated tryptophan, sugar-tryptophan adducts, and hydrolysable tryptophan products). A disclosed kit of parts for determining the ratio of “free” tryptophan to blocked tryptophan will aid the clinical laboratory and physicians in diagnosis. Treatment may consist in the administration of tryptophan, tryptophan-containing di- or tripeptides, or dietary counseling and avoidance of fructose-glucose sweetened foods and beverages.

1-15. (canceled)

16. A method for determining L-tryptophan in the feces of a subject suspected of having dietary fructose intolerance or tryptophan malabsorption or inadequate tryptophan blood levels, characterized by the steps of:
(a) collecting a defined fecal sample of said subject;
(b) extracting the fecal sample in a buffer to produce an extract of fecal amino acids and soluble fecal compounds;
(c) separating said extract from insoluble components and preparing aliquots of said extract, one for direct determination of L-tryptophan in said aliquot and one for determination of L-tryptophan in said aliquot after hydrolysis of any glycated tryptophan and any sugar-tryptophan adducts;
(d) treating an aliquot of said extract with a strong acid or base to hydrolyze any condensation products of an aldose or ketose with an amino acid or L-tryptophan;
(e) adding to said aliquots a derivatization reagent which reacts with the α-amino groups of amino acids or L-tryptophan to prepare a L-tryptophan derivative;
(f) determining the amounts of L-tryptophan derivative in said aliquots using an antibody specific for L-tryptophan derivative, and;
(g) comparing the amounts of L-tryptophan derivative in said aliquots to determine the amount and ratio of tryptophan in the subject's fecal sample that has undergone a condensation reaction with an aldose or ketose in the gastrointestinal tract and is present in the feces as a hydrolyzable L-tryptophan product.

17. The method of claim 16, comprising the step of comparing the amount of hydrolyzable L-tryptophan product in the fecal sample of said subject with the amount of hydrolyzable L-tryptophan product in the feces of a healthy subject to diagnose a dietary fructose intolerance or inadequate dietary tryptophan supply in the case of an increased amount of hydrolyzable L-tryptophan product.

18. The method of claim 16, comprising determining the ratio of hydrolyzable tryptophan product to free tryptophan in the feces.

19. The method according to claim 16, comprising a determination of tryptophan and/or hydrolyzable tryptophan product in a body fluid selected from plasma, serum, blood, and urine.

20. The method of claim 16, comprising a determination of the etiology when the subject suffers from a disorder, including digestion disorders, obstipation, diarrhea, gastrointestinal disorders, colitis, irritable bowel syndrome (IBS), inflammatory bowel disease (IBD), Crohn's disease, dermatitis, depression, insomnia, sleep disorders, migraine, fatigue syndrome, bulimia nervosa, eating disorders, anxiety, dysphoric disorders, burn-out syndrome.

21. A method according to claim 16, comprising a determination of the effective amount of one of the following: supplementary dietary tryptophan, pharmaceutical composition comprising di- and tripeptides of tryptophan, supplementary niacin, or vitamin B3.

22. Use of the method according to claim 16, wherein the derivatization reagent is selected from the group comprising:
detection reagents with a reactive component;
biotin-ε-aminocaproic acid-N-hydroxy succinimide ester,
Boc-6-aminocaproic acid N-hydroxy succinimide ester,
diiodotyrosine-beta-alanine hydroxysuccinimidyl ester,
tryptophan-β-alanine hydroxysuccinimidyl ester.

23. Use of the method according to claim 16, wherein an antibody is added which binds anti-L-tryptophan derivative antibodies, the detector antibody being conjugated to a detection group, a fluorescent or luminescent dye, an electroluminescent group or an enzyme for detection, a peroxidase.

24. Use of the method according to claim 16, wherein glycated tryptophan or sugar-tryptophan product is hydrolyzed at an elevated temperature between 60 and 100 degrees Celsius in an aqueous solution having a pH between 12 and 14.

25. Use of a kit of parts in a method according to claim 16 for determining the amount of hydrolyzable glycated tryptophan or sugar-tryptophan adduct, comprising
(a) a tryptophan derivatization reagent;
(c) antibodies which bind a tryptophan derivative obtained in (b);
(d) the tryptophan derivative of (c) as a tracer substance;
(e) one or more standard solutions of tryptophan and
(f) sugar-L-tryptophan adduct as hydrolysis control.

26. Use of a kit of parts in a method according to claim 25, comprising a microtiter plate wherein the tryptophan-derivative tracer is immobilized on a surface.

27. Use of a kit of parts in a method according to claim 25, comprising D-fructose-L-tryptophan as control.

28. Use of a kit of parts in a method according to claim 25, comprising a buffer for extraction of feces and solubilization of aromatic amino acids.

29. Use of a kit of parts in a method according to claim 25, further comprising a tool and tube system for transferring a defined amount of feces and into a vessel with extraction and stabilization buffer.

30. A method of in-vitro diagnosis of dietary fructose intolerance or excessive fructose consumption, using a kit of parts as claimed in claim 25.
